(19) United States
(12) Patent Application Publication
(10) Pub. No.: US 2025/0295467 A1
(43) Pub. Date: Sep. 25, 2025

(54) METHOD FOR CONTROLLING THE MOVEMENT OF A SURGICAL MICROSCOPE AND SURGICAL MICROSCOPE

(71) Applicant: Carl Zeiss Meditec AG, Jena (DE)

(72) Inventor: Stefan Ernsperger, Ellwangen (DE)

(21) Appl. No.: 19/231,537

(22) Filed: Jun. 8, 2025

Related U.S. Application Data
(63) Continuation of application No. PCT/EP2023/084061, filed on Dec. 4, 2023.

(30) Foreign Application Priority Data
Dec. 8, 2022 (DE) 10 2022 213 294.7

(51) Int. Cl. A61B 90/20; A61B 34/00

(52) U.S. Cl. CPC A61B 90/20; A61B 34/74; A61B 2034/742

(57) ABSTRACT

A method for controlling the movement of a surgical microscope and to a surgical microscope is provided. In a main operating mode, a movement of a microscope head in a first predetermined manner of movement is controlled by an actuation of an operating element in a first manner of actuation. An auxiliary operating mode is activatable. In the auxiliary operating mode, a movement of the microscope head in a further predetermined manner of movement is controlled by an actuation of the operating element in the first manner of actuation, and the first predetermined manner of movement, and the further predetermined manner of movement differ from one another.

### CROSS REFERENCE TO RELATED APPLICATIONS

This application is a continuation application of international patent application PCT/EP2023/084061, filed Dec. 4, 2023, designating the United States and claiming priority to German application 10 2022 213 294.7, filed Dec. 8, 2022, and the entire content of both applications is incorporated herein by reference.

### TECHNICAL FIELD

The disclosure relates to a method for controlling the movement of a surgical microscope and to a surgical microscope.

### BACKGROUND

The related art describes surgical microscopes for providing a magnified view of examination objects, in particular in medical applications. They serve, among other things, to provide a magnified view of partial regions of a body in order to enable a better visual orientation and diagnosis for a surgeon during a procedure. Also known are surgical microscopes which enable motion control, in particular of a microscope head. Such surgical microscopes include one or more drive device(s) for generating a driving force for moving parts of the surgical microscope, wherein the microscope head can be moved in the desired manner by a suitable control of the drive device(s). Examples of applications include positioning the microscope head in such a way that an optical axis of an objective of the surgical microscope takes a desired orientation, and a movement such that a reference point of the surgical microscope, e.g., a focus point, is positioned at a desired position in space. The movement of the surgical microscope can be controlled by a user, e.g., a surgeon. For this purpose, the surgical microscope may have suitable operating elements for motion control.

DE 10 2019 108 129 A1 describes such a method for motor-operated positioning of a surgical microscope. Also known is DE 10 2009 037 018 A1, which discloses a method for causing a surgical microscope to approach a position in a controlled manner. WO 2021/140513 describes a surgical system and the control of system functions. DE 10 2008 011 638 A1 describes a balancing apparatus for a surgical microscope mounted on a pivot. Described in WO 2021/252930 A1 is a robotic and digital surgical microscope and a hand-centered controller for this microscope. WO 2018/217951 A1 describes a visualization system for use during a surgical procedure. Described in DE 11 2020 000 880 T5 is a control apparatus and an ophthalmic microscope system.

### SUMMARY

It is an object of the disclosure to provide a method for controlling the movement of a surgical microscope and a surgical microscope, which simplify the control of the movement, in particular with different manners of movement, and thereby in particular ensure operational reliability.

The object is achieved by the method for controlling the movement of a surgical microscope and the surgical microscope as described herein.

For the purposes of this disclosure, a microscope refers to a device configured to provide a magnified visual view of, that is to say for microscopically imaging, an examination object. The microscope may be a light microscope, which generates an enlarged image representation by utilizing optical effects, in particular with means for beam guidance and/or beam shaping and/or beam steering, for example lenses. However, the microscope may also be a digital microscope, wherein the (magnified) image representation to be visualized by the microscope may be generated by way of an image capture device and may be displayed on an appropriate display device.

The surgical microscope includes a microscope head. The microscope head may include an objective of the surgical microscope, which can generate a real optical image representation of an examination object. The objective may include the optical elements described. The microscope head may include a housing, wherein the objective or at least parts thereof are arranged in the housing. In the housing, for example, a beam path for the microscopic imaging of the examination object may be arranged. It is conceivable that a tracking camera is also arranged in the housing, which enables optical, and, in particular, mark-supported pose determination of a target. The target may include at least one, but typically a plurality of, marks and, for example, be attached to an instrument, e.g., a surgical instrument. In this case, the housing may also include a further beam path for optical detection by way of the tracking camera, wherein the described beam paths can be formed separately from each other.

Furthermore, the surgical microscope may include a stand for holding the microscope head. The microscope head can be mechanically attached to the stand and in particular form an end effector of the stand. In this context, the stand may be configured such that it allows a movement of the microscope head in space, in particular with at least one degree of freedom, typically with six degrees of freedom, wherein a degree of freedom may be a translational or a rotational degree of freedom. A translational movement and a rotational movement, and also the corresponding degrees of freedom, can refer to a reference coordinate system. A vertical axis (z-axis) of this reference coordinate system can be oriented parallel to the gravitational force, and the corresponding vertical direction (axis direction) can be oriented counter to the gravitational force. Alternatively, the vertical axis may be oriented parallel to an optical axis of the surgical microscope, which may be an optical axis of the objective, and the corresponding vertical direction may be oriented away from the surgical microscope to an object space. A longitudinal axis (x-axis) and a transverse axis (y-axis) of the reference coordinate system may in this context span a plane that is oriented perpendicularly to the vertical axis. Furthermore, the longitudinal axis and the transverse axis may also be oriented orthogonal to each other. The longitudinal and transverse directions (axis directions) can be oriented in such a way that the axes form a Cartesian coordinate system.

The surgical microscope, in particular the stand, includes at least one drive device for moving the surgical microscope, in particular the microscope head. Such a drive device may be a servo motor, for example. Of course, the stand may also include means for transmitting forces/torques, for example gear units. Furthermore, the surgical microscope may include means for controlling the movement. Using the means of control, for example, a user can control the at least one drive device in such a way that the surgical microscope performs a desired movement in space. Thereby, the surgical microscope can be positioned, e.g., in a specified target pose in space, wherein the pose designates a position and/or orientation of the microscope head. A movement can also be controlled with a desired manner of movement, e.g., a desired direction of movement. The means of control can be configured for haptic actuation by a user/surgeon. However, this is not mandatory. Alternatively, the means can, for example, enable voice control.

The surgical microscope further includes at least one operating element. The operating element is a means for controlling the movement. The operating element may be configured for manual operation by a user, i.e., for haptic actuation. An actuation can be carried out, for example, by pressing, displacing, or rotation. As an example, and without restriction, the operating element may be configured as a joystick or as a switch, in particular as a toggle or rocker switch.

In a main operating mode, the movement of the microscope head in a first predetermined manner of movement is controlled by an actuation of an operating element in a first manner of actuation. The main operating mode can be activated, for example by actuating a corresponding activation means, which is also referred to below as further activation means. This will be explained in detail below. The manner of actuation refers to the way in which the operating element is actuated. Thus, the operating element can be configured such that it can be actuated in different manners of actuation. Actuation in different manners of actuation can be carried out, for example, when different portions of the operating element are actuated and/or the corresponding actuating force has different directions and/or different amplitudes.

For example, a movement of the microscope head in a first predetermined direction can be controlled by moving a joystick in a first direction of movement. By actuating the joystick in a direction counter to the first direction of movement, the movement of the microscope head can also be controlled in a further direction, which can be, for example, counter to the first direction. In other words, different manners of actuation may differ in at least one property of actuation, the property being, for example, a place of actuation, a direction of actuation, an intensity of actuation. The operating element may be arranged at the microscope head or on a handle of the surgical microscope. The handle can also be arranged at the microscope head or its housing. Alternatively, an operating element can also be arranged on the housing of the microscope head or elsewhere.

A manner of movement can specify at least one property of a movement. For example, a manner of movement can define a direction of movement and a type of movement. For example, a movement type can be a translational movement, a rotational movement, or a mixture thereof. A type and/or a number of degrees of freedom of movement, in particular the enabled degrees of freedom of movement, can also be defined by a manner of movement. Further, a reference point and/or a reference axis of the movement can also be defined by a manner of movement, wherein the movement in this manner of movement can then be a rotational movement around the reference point and/or the reference axis and/or a translational movement along the reference axis.

Furthermore, an auxiliary operating mode can be activated by generating an activation signal via a corresponding activation means. This activation means can also be designated the first activation means. This will be explained in detail below. If the auxiliary operating mode is activated or has been activated, a movement of the microscope head in a further predetermined manner of movement is controlled in the auxiliary operating mode by an actuation of the operating element in the first manner of actuation, with the first and the further manners of movement being different from each other. In particular, the first and the further manners of movement differ in at least one property. Merely by way of example, a translational movement can be controlled in the main operating mode when the operating element is actuated in the first manner of actuation, for example, while a rotational movement is controlled in the auxiliary operating mode by the same actuation.

In other words, the method according to an aspect of the disclosure allows different movements to be controlled by a similar actuation of the same operating element. This increases the range of (control) functions that can be controlled with the operating element. In particular, different operating elements and/or different manners of actuation of a single operating element do not need to be provided for controlling different movements, as a result of which an operation for motion control is simplified, in particular since a user does not have to actuate different operating elements or, for example, need to change hand position to this end. This also means in an advantageous manner that a space requirement and manufacturing costs of the surgical microscope are not increased with an increasing functional range.

It is conceivable that the auxiliary operating mode will be deactivated again. The deactivation can occur, for example, when the main operating mode is (re-) activated, e.g., when an activation signal of the main operating mode is generated (which can then also be a deactivation signal of the auxiliary operating mode). It is also conceivable that a corresponding deactivation signal is generated to deactivate the auxiliary operating mode. It is also possible that the auxiliary operating mode can be activated only from the activated main operating mode. Alternatively, the auxiliary operating mode can also be activatable independently of the activation status of the main operating mode.

The surgical microscope may include activation means for activating the main and auxiliary operating modes, which may be the same, but may typically be different from each other. A mode can be activated whenever the corresponding mode has not been activated. These activation means can be operable manually or acoustically, for example, or include a user interface for user input. Thus, an activation signal can be generated, e.g., by an actuation by hand or foot or by a voice command. It is also conceivable that an activation signal is generated via the operation of a graphical user interface such as a touch panel or touchscreen, e.g., by selecting a desired movement mode there.

The first and/or the further activation means can be configured, for example, as (an) operating element(s) of a hand-operated control panel or a foot-operated control panel. The hand-operated control panel can be arranged, for example, on a handle of the surgical microscope. Such an operating element can therefore be configured for actuation by a hand, in particular a finger, or a foot of a user. An operating element may be configured, for example, as a push knob or button, which generates an activation signal when pressed. The operating element can be an operating element which is freely configurable in terms of function, wherein such an operating element can be assigned various functions, e.g., by corresponding programming. Alternatively, the switching element may be a switch element which has been permanently configured in terms of function, with a predetermined function being assigned to it permanently and unalterably.

It is also conceivable that the activation of the main operating mode is carried out by the actuation of an activation means in a first manner of actuation and the activation of the auxiliary operating mode is carried out in a further manner of actuation that is different from the former. Thus, the corresponding activation means may be configured such that it can be actuated in different manners of actuation. It is also conceivable that the activation of the auxiliary operating mode takes place when the activation means is actuated for at least a predetermined time period, in particular for longer than the predetermined time period. In this case, the main operating mode can be activated if the activation means is actuated for less than the predetermined time period.

It is also conceivable that the activation means for activating the main operating mode is a switching means for switching between different main operating modes. Thus, if one of a plurality of main operating modes is activated, a further main operating mode that is different from the former can be activated if the activation means is actuated in the first manner of actuation and/or for less than the predetermined time period. This also makes it possible to activate all main operating modes in a predetermined sequence, i.e., to switch through them.

The surgical microscope may also include deactivation means for deactivating the main and auxiliary operating modes, and these may also be the same, but may typically be different from each other. The deactivation means may further be the same as or different from the activation means. An activation signal or deactivation signal for the main or auxiliary operating mode can also be generated if at least one mark element with a predetermined identity is identified based on an image, i.e., by evaluating an image representation. The image representation can be generated, for example, by the previously explained tracking camera, which may also be a constituent part of the surgical microscope or a microscopy system. Of course, however, it is also conceivable that the image representation which is evaluated for identification is generated by an image capture device of the surgical microscope for microscopic imaging. It is conceivable, for example, that the main operating mode is activated or deactivated when a first identity is identified and the auxiliary operating mode is activated or deactivated when a different, further identity is identified. Activation of an operating mode can also result in deactivation of the previously activated operating mode. In this case, the activation means of one operating mode thus forms the deactivation means for deactivating another operating mode.

In an exemplary embodiment, the further manner of movement is a translational movement along an optical axis of the microscope head, in particular in or counter to the axis direction, which can be oriented from the microscope head to the object space. Thus, in the auxiliary operating mode, the first manner of actuation of the operating element can control a translational movement in a first direction along the optical axis and a further manner of actuation can control a translational movement in the opposite direction. It has been shown that a translational movement along the optical axis is less often desired by a user compared with other manners of movement. By assigning this manner of movement to the auxiliary operating mode, other, more frequently desired manners of movement can advantageously be assigned to the main operating mode and do not require any additional activation compared with the auxiliary operating mode. This in turn advantageously simplifies the operation of the surgical microscope, and at the same time the explained translational movement is made possible.

In a further exemplary embodiment, the auxiliary operating mode is deactivated after a predetermined time period of inactivity has expired. The time period of inactivity refers to a time period during which the operating element is not actuated. For example, it can be 5 seconds. Thus, if the auxiliary operating mode has been activated and the operating element is not actuated or not actuated according to a selected manner of actuation or according to several selected manners of actuation, the auxiliary operating mode is deactivated. It is conceivable that after the auxiliary operating mode has been deactivated, the surgical microscope will be set to a status in which both the main operating mode and the auxiliary operating mode are deactivated. In this case, the main operating mode must first be activated again for motion control. Typically, however, the main operating mode is activated after or with the deactivation of the auxiliary operating mode. Alternatively, the time period of inactivity may be a time period during which-irrespective of an actuation of the operating element-no movement takes place, which is to say in particular no movement command is generated. It is therefore conceivable that no movement occurs despite actuation of the operating element in the auxiliary operating mode, e.g., due to a defect or a collision. It is also possible to then switch automatically to the main operating mode. This advantageously increases the operational reliability of the surgical microscope, in particular if the further manner of movement is a translational movement along the optical axis, since a risk of collision with a patient or further surgical equipment is then reduced.

Alternatively, the auxiliary operating mode is deactivated when a trajectory limit of the movement is reached in the auxiliary operating mode. If, for example, a movement along the optical axis is controlled in the auxiliary operating mode, the auxiliary operating mode can be deactivated when a focus limit is reached. For example, this limit can be a limit of a range for permissible focus positions, wherein permissible focus positions can be predetermined. This also advantageously increases the operational reliability of the surgical microscope.

In a further exemplary embodiment, the main operating mode is activated after the predetermined time period of inactivity has expired. This has already been explained above. This advantageously increases the operational reliability according to the previous explanations, while at the same time user-friendliness is also increased, since after the deactivation of the auxiliary operating mode, a movement can still be controlled without re-activation of the main operating mode being necessary.

In a further exemplary embodiment, the first manner of movement defines a translational movement in a plane that is oriented perpendicularly to the optical axis. Alternatively, the first manner of movement defines a rotational movement. The defined rotational movement can be performed, for example, around a point on the optical axis, in particular around a focus point. Alternatively, the rotational movement can be performed around a reference point of the microscope head. The reference point may be arranged, for example, on one or more axes about which a microscope head mounted on the stand can rotate, in particular in an intersection of these plurality of axes of rotation. In particular, a direction of movement of the translational movement or the rotational movement can also be defined by the first manner of movement. Observations show that the mentioned manners of movement are more often desired by a user compared with in particular the translational movement along the optical axis. By assigning this manner of movement to the main operating mode, other, less frequently desired manners of movement can advantageously be assigned to the auxiliary operating mode. This in turn advantageously simplifies the operation of the surgical microscope, while at the same time allowing the translational movement along the optical axis.

In a further exemplary embodiment, an activation signal for activating the auxiliary operating mode is haptically generated. The surgical microscope may to this end include suitable activation means (first activation means), for example a manually actuable activation means such as a push button, a switch or a different activation means for manual actuation. This has already been explained above. Such an activation means may be arranged in particular at the microscope head, in particular on its housing, or on a handle. This advantageously results in a simple and reliable activation of the auxiliary operating mode. Alternatively, the activation signal is generated acoustically, for example via a voice signal. In this case, the surgical microscope or a microscopy system including the surgical microscope may include means for speech-based activation, in particular at least one microphone and an evaluation device for evaluating acoustic signals. Then, depending on the evaluation of an acoustic signal, the activation signal for activating the auxiliary operating mode can be generated.

This advantageously results in simplified operation of the surgical microscope, in particular a simplified activation of the auxiliary operating mode.

In a further exemplary embodiment, the operating element is configured for actuation in a plurality of manners of actuation, wherein the auxiliary operating mode is activated for exactly one or more, but not all, selected manner(s) of actuation. In other words, it is conceivable that a movement of the microscope head in a first predetermined manner of movement is controlled in the activated main operating mode by an actuation of the operating element in a first manner of actuation, wherein the movement of the microscope head in a second predetermined manner of movement, which is different from the first manner of movement, is controlled by the actuation of the operating element in a further manner of actuation. If the auxiliary operating mode is then activated, the movement of the microscope head in a further predetermined manner of movement, which is at least different from the first, but typically also from the second, manner of movement, can be controlled by the actuation of the operating element in the first manner of actuation. However, by actuating the operating element in the further manner of actuation, the movement of the microscope head in the second predetermined manner of movement described above is controlled in the activated auxiliary operating mode. This advantageously results in a further improvement of the functionality of the surgical microscope, since different manners of movement of the main and auxiliary operating modes can be combined by different manners of actuation.

In a further exemplary embodiment, the operating element is configured as a joystick or as a rocker switch. This results in a simple and cost-effective production of the surgical microscope.

In a further exemplary embodiment, an activation signal for activating a main operating mode is generated with a further activation means, which is different from the first activation means for activating the auxiliary operating mode. There may be a plurality of, e.g., three, main operating modes which differ from one another, wherein each of these modes can be activated, or switching between these modes can be effected, with the further activation means. For example, different main operating modes can be activated with a graphical user interface. This results in an advantageous manner in reliable operation of the surgical microscope, in particular an activation of the operating modes. Different main operating modes can define in particular manners of movement which differ from one another, with which the movement of the surgical microscope is controlled when the operating element is actuated in a first manner of actuation.

Further provided is a surgical microscope including at least one microscope head, at least one operating element for controlling the movement of the microscope head, and at least one control device. Thus, the surgical microscope is configured in such a way that a method according to any of the exemplary embodiments described in this disclosure can be carried out with the surgical microscope.

Also provided is a microscopy system including the surgical microscope. The microscopy system may include further activation means for activating a main operating mode and first activation means for activating the auxiliary operating mode.

### BRIEF DESCRIPTION OF THE DRAWINGS

The disclosure will now be described with reference to the drawings wherein:

FIG. 1 shows a schematic view of a surgical microscope according to an exemplary embodiment of the disclosure,

FIG. 2 shows a schematic view of manners of movement of a main operating mode of a surgical microscope,

FIG. 3 shows a schematic illustration of further manners of movement of a main operating mode of a surgical microscope,

FIG. 4 shows a schematic illustration of further manners of movement of a main operating mode of a surgical microscope,

FIG. 5 shows a schematic illustration of manners of movement of an auxiliary operating mode of a surgical microscope,

FIG. 6 shows a schematic flowchart of a method according to a first exemplary embodiment of the disclosure,

FIG. 7 shows a schematic flowchart of a method according to a further exemplary embodiment of the disclosure,

FIG. 8 shows a schematic flowchart of a method according to the disclosure in accordance with a further embodiment,

FIG. 9 shows a schematic flowchart of a method according to a further exemplary embodiment of the disclosure, and

FIG. 10 shows a schematic illustration of an operating element.

### DESCRIPTION OF EXEMPLARY EMBODIMENTS

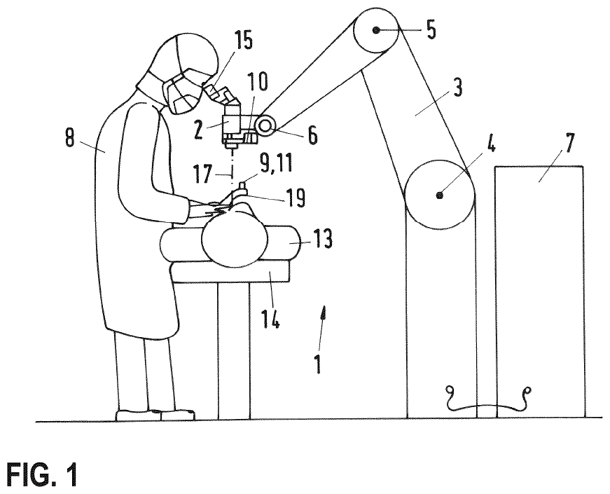

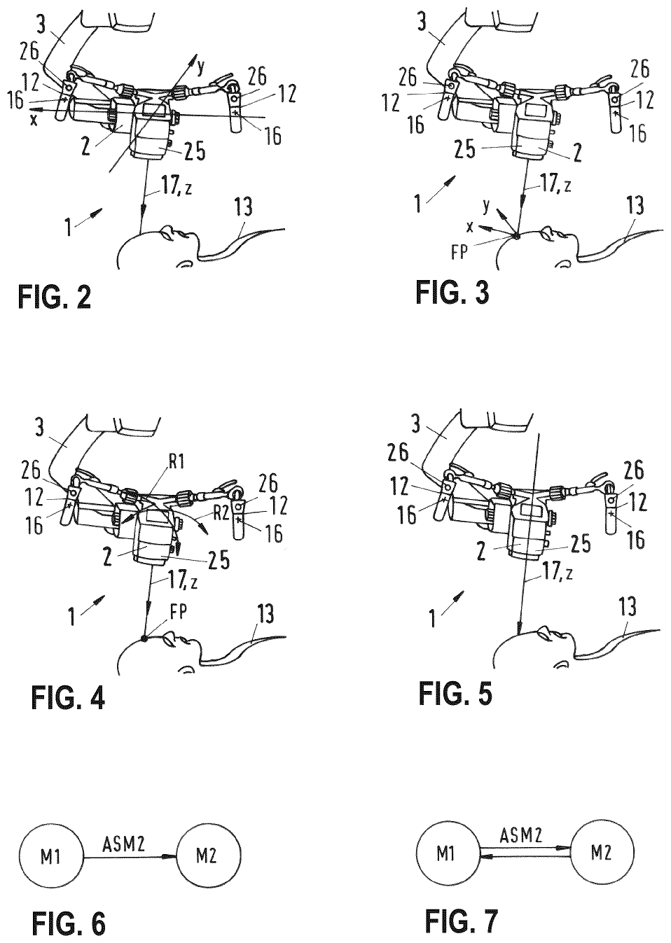

Identical reference signs hereinafter denote elements having identical or similar technical features. FIG. 1 illustrates a surgical microscope 1 according to the disclosure during use in a surgical environment. The surgical microscope 1 includes a microscope head 2, which is arranged at a free end of a stand 3 for holding the microscope head 2. The stand 3 allows a controlled movement of the microscope head 2 to change the pose, i.e., the position and/or orientation, of the microscope head 2 and thus also of an optical axis 17 of an objective (not shown) of the surgical microscope 1, which can be arranged in a housing 25 of the microscope head 2 (see, e.g., FIG. 2). The stand 3 shown is an exemplary kinematic structure for holding and moving the microscope head 2. A person skilled in the art will of course know that other kinematic structures may also be used. Drive devices (not depicted) of the stand 3 may enable a rotational movement of movable parts of the stand 3 about axes of rotation 4, 5, 6. The figure also illustrates a control device 7 that serves to control the drive devices and thus the movement. To this end, the control device 7 can be connected for signal and/or data exchanges to the drive devices. Also illustrated is a patient 13 lying on an operating table 14. It further illustrates that the surgical microscope 1, more precisely the microscope head 2, includes at least one eyepiece 15 or an optical viewer into which the user 8, e.g., a surgeon, looks in order to thereby view a partial region of the patient 13, in particular in an enlarged manner. FIG. 1 does not show handles 12 (see FIG. 2) of the microscope head 2.

The surgical microscope 1 moreover includes a tracking camera 10 for detecting a pose of an instrument 19 that can be held and moved by the user 8. In this case, a target 9 with at least one mark 11 can be attached to the instrument 19, wherein the pose of the target 9 can be determined based on an image representation of the target 9 captured by the tracking camera 10 and the pose of the instrument 19 can also be determined on the basis of the fixed arrangement of the target 9 on the instrument 19. A mark 11 or the target 9 may have an, in particular unique, identity, which in particular can also be determined on the basis of an image. If an identity is detected based on an image, an operating mode assigned to the identity can thus be activated or deactivated.

FIG. 2 shows a schematic illustration of manners of movement of a main operating mode M1 (see FIG. 6) of a surgical microscope 1 with a microscope head 2, which is attached to a stand 3. At the microscope head 2, two handles 12 are attached which protrude from a housing 25 of the microscope head 2. A surgeon can grip these handles 12 with the hands and move or position the microscope head 2 in space by a hand movement in the desired manner. On the handles 12, an operating element 16 for actuation by the user, in particular with the thumb or another finger, can be arranged in each case. By actuating the operating element 16, a movement of the microscope head 2 in a first predetermined manner of movement can, in a main operating mode M1, be controlled. FIG. 2 shows a longitudinal translation axis x and a transverse translation axis y and also an optical axis 17, which corresponds to a vertical axis z. The axis directions of these axes x, y, z are represented by arrows. The longitudinal and transverse translation axes x, y are oriented perpendicularly to each other and perpendicularly to the optical axis 17. The axes x, y, 17 intersect in a reference point of the microscope head 2. It may lie in particular on at least one axis of rotation of a swivel joint, via which the microscope head 2 is attached to a movable element of the stand 3.

By actuating one of the operating elements 16 shown in various manners of actuation, the movement of the microscope head 2 can be controlled in a main operating mode M1 in and counter to the longitudinal translational direction and in and counter to the transverse translational direction. If an auxiliary operating mode M2 is activated, the movement of the microscope head 2 in and counter to the direction of the optical axis 17 can be effected by an actuation of the operating element 16 in one or more modes of actuation. A first activation means 26 configured as a push button for actuation by the user, in particular with the thumb or another finger, can also be arranged on the handles 12 in each case. By actuating the first activation means 26, an activation signal ASM2 for activation of the auxiliary operating mode M2 can be generated.

FIG. 3 shows a schematic illustration of manners of movement of a main operating mode M1 (see FIG. 6) of a surgical microscope 1. In contrast to the exemplary embodiment shown in FIG. 2, a longitudinal axis x, a transverse axis y and a vertical axis z are shown, which intersect in a focus point FP. The vertical axis z is the optical axis 17 of the surgical microscope 1 and oriented from the microscope head 2 toward a patient 13. A reference point of the microscope head 2 which can lie on a rotational axis is not shown, wherein the microscope head 2 attached to the stand 3 can rotate about this axis of rotation. In particular, the reference point may lie on an intersection of two or more than two such axes of rotation. In a main operating mode M1 (see FIG. 6), a rotational movement of the microscope head 2 around the reference point and about an axis that is parallel to the transverse axis y and that passes through the reference point can be controlled by actuation of one of the operating elements 16 in a first manner of actuation. By actuation in a further manner of actuation, a rotational movement of the microscope head 2 around the reference point and about an axis that is parallel to the longitudinal axis x and that passes through the reference point can be controlled. If an auxiliary operating mode M2 is activated, a movement of the microscope head 2 along or counter to the vertical axis z can be controlled by an actuation of the operating element 16 in the first manner of actuation.

FIG. 4 shows a schematic illustration of manners of movement of a main operating mode M1 (see FIG. 6) of a surgical microscope 1. In contrast to the exemplary embodiment shown in FIG. 2, a longitudinal axis (not shown), a transverse axis (not shown) and a vertical axis z intersect in a focus point FP. The vertical axis z is the optical axis 17 of the surgical microscope 1 and oriented from the microscope head 2 toward a patient 13. In a main operating mode M1 (see FIG. 6), a rotational movement R1 of the microscope head 2 around the focus point FP and about the longitudinal axis can be controlled by actuation of one of the operating elements 16 in a first manner of actuation. By actuation in a further manner of actuation, a rotational movement R2 of the microscope head 2 around the focus point and the transverse axis y is controllable. If an auxiliary operating mode M2 is activated, a movement of the microscope head 2 along and counter to the vertical axis z can be controlled by an actuation of the operating element 16 in the first manner of actuation.

FIG. 5 shows a schematic illustration of manners of movement of an auxiliary operating mode M2 (see FIG. 6) of a surgical microscope 1. If the auxiliary operating mode M2 is activated, a movement of the microscope head 2 along or counter to the vertical axis z, which corresponds to the optical axis 17 of the microscope head 2, can be controlled by an actuation of an operating element 16 in a first manner of actuation.

FIG. 6 shows a schematic flowchart of a method according to an exemplary embodiment of the disclosure. It shows that a main operating mode M1 is activated. In this main operating mode M1, a movement of a microscope head 2 in a first predetermined manner of movement is controlled by an actuation of an operating element 16 (see, e.g., FIG. 2) in a first manner of actuation. By generating an activation signal ASM2, an auxiliary operating mode M2 is activated. In the auxiliary operating mode M2, i.e., in the activated status, a movement of the microscope head 2 in a further predetermined manner of movement, which is different from the first manner of movement, is controlled by an actuation of the operating element 16 in the first manner of actuation. The activation signal ASM2 can be generated with a first activation means 26. Exemplary activation means and activation methods were described previously.

FIG. 7 shows a schematic flowchart of a method according to a further exemplary embodiment of the disclosure. In contrast to the exemplary embodiment shown in FIG. 6, the main operating mode M1 is activated proceeding from the activated auxiliary operating mode M2 after a predetermined time period of inactivity has expired. When activating the main operating mode M1 proceeding from the activated auxiliary operating mode M2, the auxiliary operating mode M2 is deactivated at the same time. As an alternative to the expiration of the predetermined time period of activity, a deactivation signal DASM2 can also be generated, which leads to the activation of the main operating mode M1 and to the deactivation of the auxiliary operating mode M2. Exemplary deactivation means and deactivation methods have also been described previously.

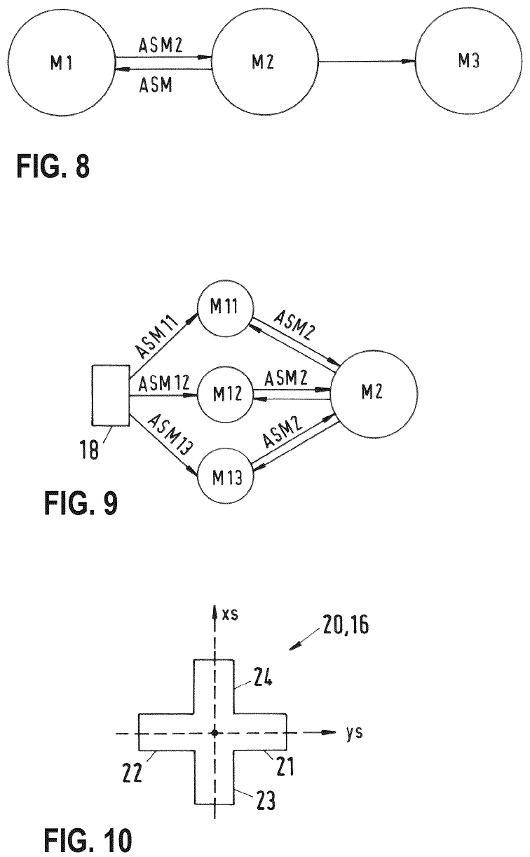

FIG. 8 shows a schematic flowchart of a method according to a further exemplary embodiment of the disclosure. In contrast to the exemplary embodiment shown in FIG. 7, the main operating mode M1 is activated proceeding from an activated auxiliary operating mode M2 when generating an activation signal ASM1 for the main operating mode M1, for example by actuating a suitable further activation means 18 (see FIG. 9). The auxiliary operating mode M2 is also deactivated after a predetermined time period of inactivity has expired, wherein upon such a deactivation the surgical microscope is set to a status M3 in which neither the main operating mode M1 nor the auxiliary operating mode M2 is activated. It is of course conceivable that in the activated main or auxiliary operating mode M1, M2, a deactivation signal (not shown) is generated with which the surgical microscope 1 is also set to this status M3.

FIG. 9 shows a schematic flowchart of a method according to a further exemplary embodiment of the disclosure. A further activation means 18 for activating a main operating mode M11, M12, M13 is shown in FIG. 9. This further activation means 18 may, for example, be configured as or include a graphical user interface. Of course, the further activation means 18 can also take another form, e.g., of an operating element of a hand-operated or foot-operated control panel or of a voice-controlled activation means. As explained above, the further activation means 18 can also be formed by the first activation means 26 (see, e.g., FIG. 2). By a corresponding actuation/control of the further activation means 18, various activation signals ASM11, ASM12, ASM13 can be generated, which lead to the activation of a first main operating mode M11, a second main operating mode M12, or third main operating mode M13. These can differ in particular with the manners of movement in which a movement of the microscope head 2 is controlled when actuating an operating element 16 (see FIG. 2). Proceeding from each of the main operating modes M11, M12, M13 activated in this way, the auxiliary operating mode M2 can then be activated by generating an activation signal ASM2. From this activated auxiliary operating mode M2, the previously activated main operating mode M11, M12, M13 can be activated again, either by generating a corresponding activation signal or after a predetermined time period of inactivity has expired.

FIG. 10 shows a schematic top view of an operating element 16 configured as a cross rocker switch 20. A longitudinal axis xs of the switch and a transverse axis ys of the switch are shown, which span a switch-specific coordinate system, wherein an origin of this coordinate system is arranged in a geometric center of gravity of the cross rocker switch 20. Actuation in a first manner of actuation of this cross rocker switch 20 can be carried out by pressing on a first leg 21 of the cross rocker switch 20, which is then tilted about the longitudinal axis xs of the switch in a mathematically positive direction relative to the axis direction shown. Actuation in a second manner of actuation of this cross rocker switch 20 can be carried out by pressing on a second leg 22 of the cross rocker switch 20, which is then tilted about the longitudinal axis xs of the switch in a mathematically negative direction relative to the axis direction shown.

Actuation in a third manner of actuation of this cross rocker switch 20 can be carried out by pressing on a third leg 23 of the cross rocker switch 20, which is then tilted about the transverse axis ys of the switch in a mathematically positive direction relative to the axis direction shown. Actuation in a fourth manner of actuation of this cross rocker switch 20 can be carried out by pressing on a fourth leg 24 of the cross rocker switch 20, which is then tilted about the transverse axis ys of the switch in a mathematically negative direction relative to the axis direction shown.

If a main operating mode M1 (see, e.g., FIG. 6) is activated, the actuation of the cross rocker switch 20 in the first manner of actuation can control a movement of the microscope head 2 in the opposite direction of a longitudinal axis x, wherein the latter—as shown, for example, in FIG. 3—runs through a focus point FP and is oriented perpendicularly to a vertical axis z, wherein the vertical axis z in turn is oriented parallel to the optical axis 17. Further, the actuation of the cross rocker switch 20 in the second manner of actuation can control a movement in the direction of the longitudinal axis x. The actuation of the cross rocker switch 20 in the third manner of actuation can control a movement in the opposite direction of a transverse axis y, which also runs through the focus point FP and forms a Cartesian coordinate system with the longitudinal and vertical axes x, z. The actuation of the cross rocker switch 20 in the fourth manner of actuation can control a movement in the direction of the transverse axis y.

If, on the other hand, an auxiliary operating mode M2 is activated, then the actuation of the cross rocker switch 20 in the first manner of actuation can control a movement of the microscope head 2 in the direction of the vertical axis z. Further, the actuation of the cross rocker switch 20 in the second manner of actuation can control a movement counter to the direction of the vertical axis z.

When the cross rocker switch 20 is actuated in the third manner of actuation and in the fourth manner of actuation, no movement of the microscope head 2 can be controlled in the activated auxiliary operating mode M2, and so the actuation cannot cause motion control. Alternatively, the function of the actuation can be maintained in the activated main operating mode M1. Then, the actuation of the cross rocker switch 20 in the third manner of actuation can thus control a movement counter to the direction of the transverse axis y, and the actuation of the cross rocker switch 20 in the fourth manner of actuation can control a movement in the direction of the transverse axis y.

### LIST OF REFERENCE NUMERALS

1 Surgical microscope
2 Microscope head
3 Stand
4, 5, 6 Axes of rotation
7 Control device
8 User
9 Target
Tracking camera
11 Mark
12 Handle
13 Patient
14 Operating table
Eyepiece
16 Operating element
17 Optical axis
18 Further activation means
19 Instrument
Cross rocker switch
21, 22, 23, 24 Leg of the cross rocker switch
25 Housing
26 First activation means
M1, M11, M12, M13 Main operating mode, main operating modes
M2 Auxiliary operating mode
M3 Status
ASM1, ASM2
ASM11, ASM12, ASM13 Activation signal
x, xs Longitudinal axis
y, ys Transverse axis
z Vertical axis
FP Focus point
R1, R2 Rotational movement

What is claimed is:

1. A method for controlling a movement of a surgical microscope, the method comprising:
controlling the movement of a microscope head in a first predetermined manner of movement in a main operating mode by an actuation of an operating element in a first manner of actuation;
activating an auxiliary operating mode; and
in the auxiliary operating mode, controlling the movement of the microscope head in a further predetermined manner of movement by the actuation of the operating element in the first manner of actuation, wherein the first predefined manner of movement and the further predefined manner of movement are different from one another.

2. The method as claimed in claim 1, wherein the further predefined manner of movement is a translational movement along an optical axis (17) of the microscope head (2).

3. The method as claimed in claim 1, further comprising:
deactivating the auxiliary operating mode after an expiration of a predetermined time period of inactivity or when a trajectory limit of the movement in the auxiliary operating mode is reached.

4. The method as claimed in claim 3, further comprising:
activating the main operating mode after the expiration of the predetermined time period of inactivity.

5. The method as claimed in claim 1, wherein the first predefined manner of movement defines a translational movement in a plane oriented perpendicularly to an optical axis or a rotational movement.

6. The method as claimed in claim 1, further comprising:
generating an activation signal for activating the auxiliary operating mode haptically or acoustically.

7. The method as claimed in claim 1, wherein the operating element is configured to actuate in a plurality of manners of actuation, and
wherein the auxiliary operating mode is activated for exactly one or more, but not all, selected manner(s) of actuation.

8. The method as claimed in claim 1, wherein the operating element is configured as a joystick or as a rocker switch.

9. The method as claimed in claim 1, wherein an activation signal for activating the main operating mode is generated with a further activation means, which is different from the first activation means for activating the auxiliary operating mode.

10. The method as claimed in claim 1, wherein the microscope head is mechanically attached to a stand having at least one drive device configured to move the microscope head.

11. A surgical microscope comprising:
at least one microscope head;
at least one operating element for controlling the movement of the microscope head; and
at least one control device,
wherein the surgical microscope is configured such that:
in a main operating mode, a movement of the microscope head in a first predetermined manner of movement is controlled by an actuation of the operating element in a first manner of actuation,
an auxiliary operating mode can be activated, and
in the auxiliary operating mode, a movement of the microscope head in a further predetermined manner of movement is controlled by the actuation of the operating element in the first manner of actuation, wherein the first predetermined manner of movement and the further predetermined manner of movement are different from one another.

12. The surgical microscope as claimed in claim 11, wherein the microscope head is mechanically attached to a stand having at least one drive device for moving the microscope head.
